(19) United States
(12) Patent Application Publication
(10) Pub. No.: US 2016/0095723 A1
(43) Pub. Date: Apr. 7, 2016

(54) ABOVE-THE-KNEE AND BELOW-THE-KNEE PROSTHETIC COVERS

(71) Applicant: James Devito, Treasure Island, FL (US)

(72) Inventor: James Devito, Treasure Island, FL (US)

(21) Appl. No.: 14/820,349

(22) Filed: Aug. 6, 2015

Related U.S. Application Data
(63) Continuation-in-part of application No. 29/529,580, filed on Jun. 9, 2015.
(60) Provisional application No. 62/060,061, filed on Oct. 6, 2014.

(51) Int. Cl. A61F 2/78

(52) U.S. Cl. CPC A61F 2/78; A61F 2002/608

(57) ABSTRACT

A cover for a prosthetic leg having an outer layer, an inner layer and a foam insert. The outer layer has at least two side edges and an inner surface. The inner layer is permanently attached to the outer layer at the two side edges so as to form a pocket between the inner surface of the outer layer and the inner layer. The foam insert is shaped as a partial human leg, and has an inner cavity and an outer surface. The foam insert is positioned inside the formed pocket such that its inner cavity is located adjacent to the inner layer, its outer surface faces the inner surface of the outer layer, and the entire foam insert is located below the top edge of the inner layer. Further, the cover includes a closing mechanism attached to the two side edges to selectively and securely attach the two side edges to each other.

### RELATED APPLICATIONS

This application claims priority to a U.S. Provisional Patent Application Ser. No. 62/060,061 filed on Oct. 6, 2014 and incorporated herewith by reference in its entirety. This is a continuation in part of U.S. Design Application Ser. No. 29/529,580 entitled BELOW-THE-KNEE PROSTHETIC COVER, filed Jun. 9, 2015 (pending).

### BACKGROUND OF THE INVENTION

This application and its disclosure generally relate to the field of prosthetics.

A prosthetic limb is used to replace an amputated portion of a limb and to help restore the amputees ability to use the limb. A prosthetic leg for a lower extremity typically will include artificial foot and ankle components coupled to an upright assembly which, in turn, is coupled to a custom-fitted socket assembly. If the amputation is above the knee, the upright assembly typically includes an artificial knee joint. Often, a cosmetic cover is fabricated to cover the prosthetic limb, such as to cover a prosthetic leg from the socket downward to the artificial foot.

In one conventional method for fabricating a cosmetic cover for a prosthetic leg, the artificial foot is first removed. Then, a cosmetic cover blank is obtained having the shape of a tapered cylinder with a constant wall thickness. The cosmetic cover blank is made of a foam material such as PVC (polyvinyl chloride) foam, EVS foam, ethafoam, or polyethylene foam. The cosmetic cover blank is heated to expand to a point where it will slide over the prosthetic limb. It is noted that differing diameters cause the proximal end of the cosmetic cover blank (i.e., the end of the cosmetic cover blank designed to be closest to the residual limb) to expand more than the distal end of the cosmetic cover blank (i.e., the end of the cosmetic cover blank designed to be farthest from the residual limb). Then, the heated and expanded cosmetic cover blank is slid onto the prosthetic leg. The socket has a bonding agent to bond the inside surface of the cosmetic cover blank to the outside surface of the socket, and a vacuum is applied to draw the cover onto the prosthetic leg as it cools. After cooling, the artificial foot is reattached, and the distal surface of the cosmetic cover blank is bonded to the proximal surface of the artificial foot. The cosmetic cover blank is then manually “shaved” to mimic the anatomy of a sound limb using at least the dimensions of the circumference of the ankle and calf section of the amputees sound leg. A spray skin finish or cosmetic hose is then applied to the covered prosthetic leg.

Generally, all currently available covers are fabricated as a reverse mold of the limb they are designed to cover. The shape of the inner surface of such conventional cover is made to be the exact inverse of the shape of the surface of the prosthetic leg it is covering, so the cover fits snug on the prosthetic leg and does not spin. Such covers are typically made of hard material, for example, hard plastic or metal.

### SUMMARY

In one general aspect, the present invention provides the Above-the-Knee (“AK”) and Below-the-Knee (“BK”) covers which are used as covers for an artificial limb.

Generally, the covers include an outer layer, an inner layer and a foam insert. The outer layer has at least two side edges and an inner surface. The inner layer is permanently attached to the outer layer at the two side edges so as to form a pocket between the inner surface of the outer layer and the inner layer. The foam insert is shaped as a partial human leg, and has an inner cavity and an outer surface. The foam insert is positioned inside the formed pocket such that its inner cavity is located adjacent to the inner layer, its outer surface faces the inner surface of the outer layer, and the entire foam insert is located below the top edge of the inner layer. Further, the cover includes a closing mechanism attached to the two side edges to selectively and securely attach the two side edges to each other.

Because of the simplicity of the artificial limbs and the fact that the polyurethane foam can conform to any shape, the disclosed covers provide universal leg covers. Currently, the disclosed design fits about 90% of all above-the-knee artificial limbs on the market. No more need to buy a cover customized for a particular leg. Further, the disclosed design eliminates all the time that prosthetists spend shaping a solid and permanent cover by providing an easy on easy off the shelf universal shape cover.

The above aspects, advantages and features are of representative embodiments only. It should be understood that they are not to be considered limitations on the invention as defined by the claims. Additional features and advantages of the invention will become apparent in the following description, from the drawings, and from the claims.

### BRIEF DESCRIPTION OF THE DRAWINGS

The invention is illustrated by way of example and not limitation and the figures of the accompanying drawings in which like references denote like or corresponding parts, and in which:

FIG. 1 shows a typical prosthetic leg without any cover;

FIG. 2 shows a cover for the prosthetic leg in accordance with a first embodiment of the present invention;

FIG. 3 shows an open view of the cover shown in FIG. 2;

FIG. 4 shows a schematic view of the foam insert;

FIG. 5 shows a schematic view of the outer surface of the foam insert;

FIG. 6 shows a closed top view of the cover shown in FIG. 2;

FIG. 7 shows a side view of a cover for the prosthetic leg in accordance with a second embodiment of the present invention;

FIG. 8 shows a rear view of the cover shown in FIG. 7;

FIG. 9 shows a schematic view of a cover for the prosthetic leg in its open position; and

FIG. 10 shows a cover for the prosthetic leg in accordance with a third embodiment of the present invention;

### DETAILED DESCRIPTION OF THE PREFERRED EMBODIMENT AND THE DRAWINGS

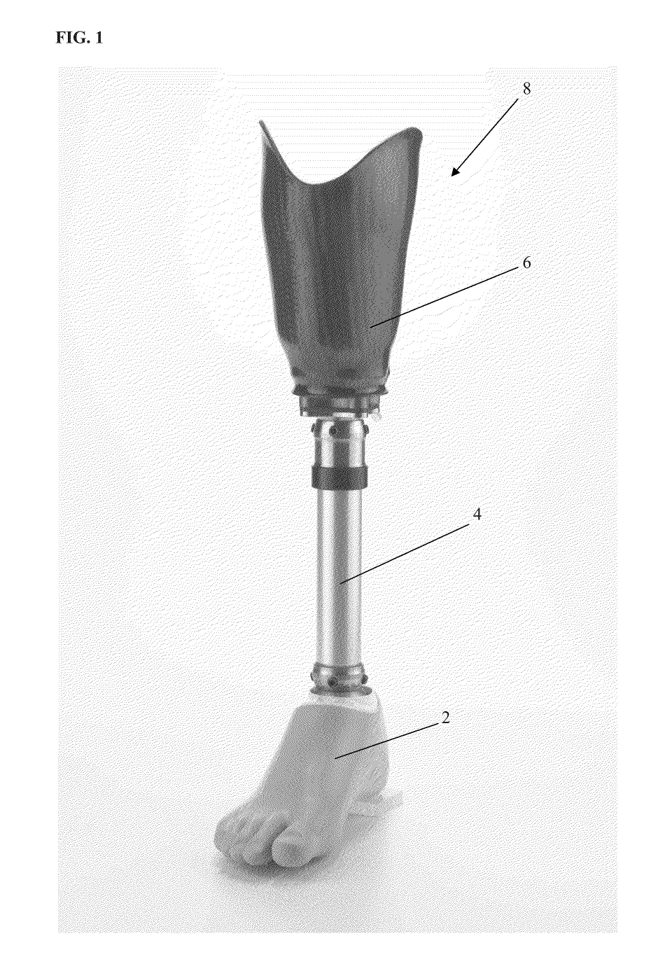

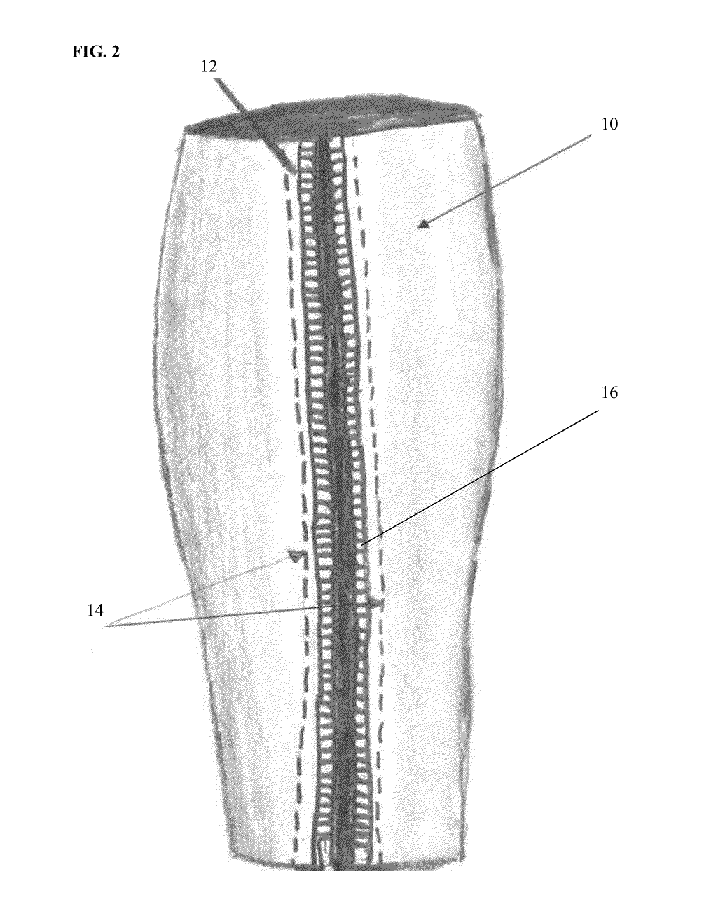

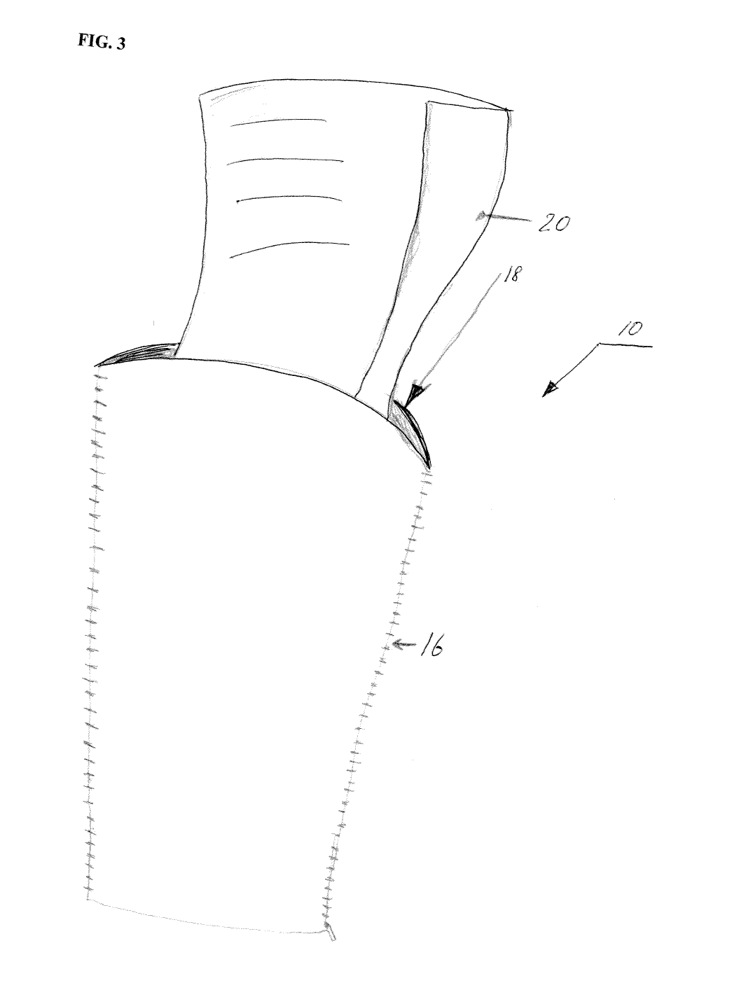

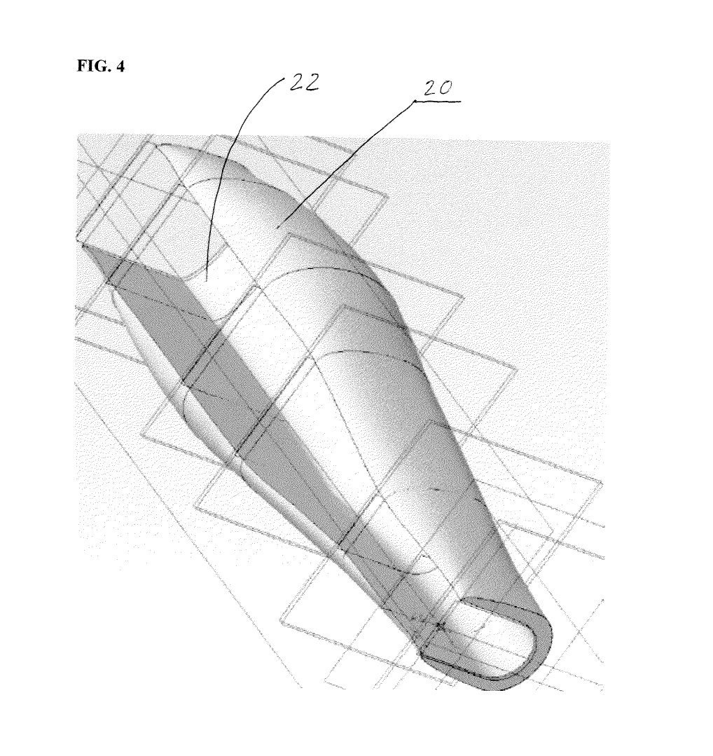

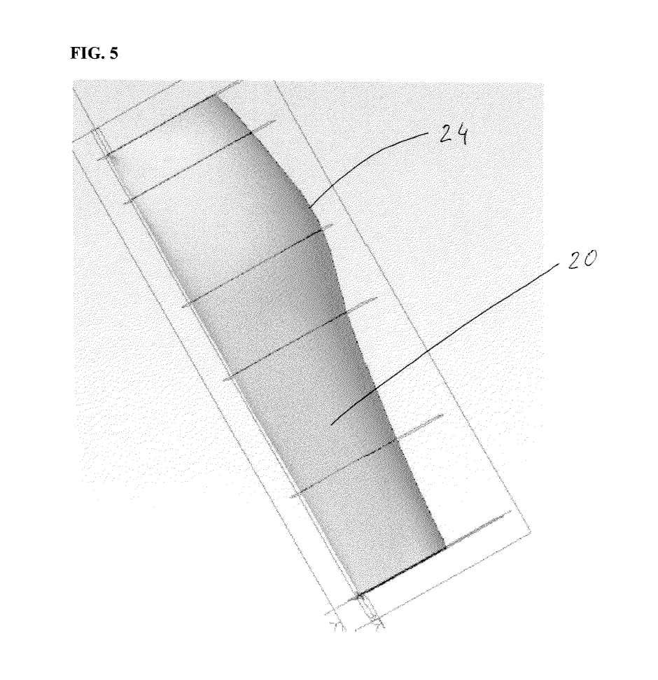

Referring to FIG. 1, a traditional prosthetic leg 8 typically includes a foot portion 2, a pylon 4 and a socket 6. In accordance with the preferred embodiment of the present invention, a cover 10 for the prosthetic leg 8 is provided, as shown in FIG. 2. Cover 10 is a substantially tubular member which includes a vertical split opening 12 along the entire length of its frontal face. A zipper 16 is preferably sewn into the split opening along its two edges 14. FIG. 3 shows cover 10 in its open position. As shown in FIG. 3, an internal pocket 18 is formed within the cover. An insert 20 formed in the shape of a normal, healthy human calf and upper ankle is inserted into the internal pocket 18. The insert is preferably made from Plastazote or any other suitable type of foam. An example of the foam insert 20 is shown in FIGS. 4-5. Foam insert 20 is formed with an interior cavity 22 shaped to fit snugly over the pylon 4 and socket 6. An exterior surface 24 of the foam insert is shaped to represent a healthy, normal calf muscle proportionate to the size of a particular patient. As shown in FIGS. 2-5, cover 10 and foam insert 20 are anatomically constructed and cut to give the illusion of a leg which is larger at a calf and tapers down to a smaller ankle.

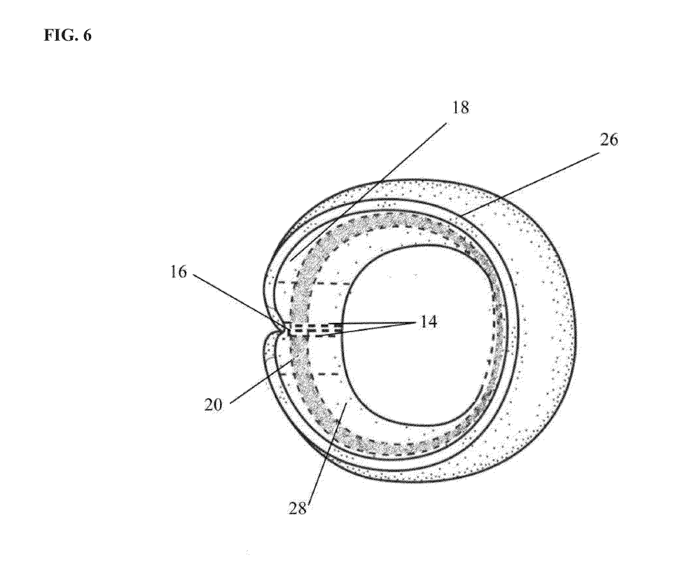

The view from the top of the assembled cover is shown in FIG. 6. As can be seen in the figure, pocket 18 is formed between an outer layer 26 and inner layer 28. The layers are preferably connected to each along the edges 14. In use, a patient will first insert the foam insert 20 into the pocket 18, then place the assembled cover 10 around the pylon 4 and socket 6, and then secure the assembled cover by zipping up the zipper 16.

Where the cover extends above the knee, it is referred to as the Above-the-Knee (“AK”) cover. The AK cover preferably has four lengths sizes, i.e., Extra Tall, Tall, Medium and Short. The sizes are defined by the measurement taken from the top of the foot portion to the bend in the knee. There are also two preferred widths (as measured at the socket) of the AK cover, i.e., Regular at 14½ inches and Wide at 16½ inches. Where the cover reaches only to the area below the knee, it is referred to as the Below-the-Knee (“BK”) cover. The BK covers preferably have three universal sizes (lengths) and four universal widths. To correctly determine the size of the cover, the user will first measure the distance between his/her foot portion 2 and the end of the pylon 4, and then add 4 inches. The resulting measurement determines the size as follows: Short is 10 to 11 inches, Medium is 11 to 13 inches, and Tall is 13 to 14 inches. To determine the correct width, the user will measure the diameter of the socket 4 inches above the end of the pylon. The resulting measurement determines the width as follows: Small is 10 to 12 inches. Medium is 12 to 14 inches, Large is 14 to 16 inches and X-Large is 16 to 18 inches.

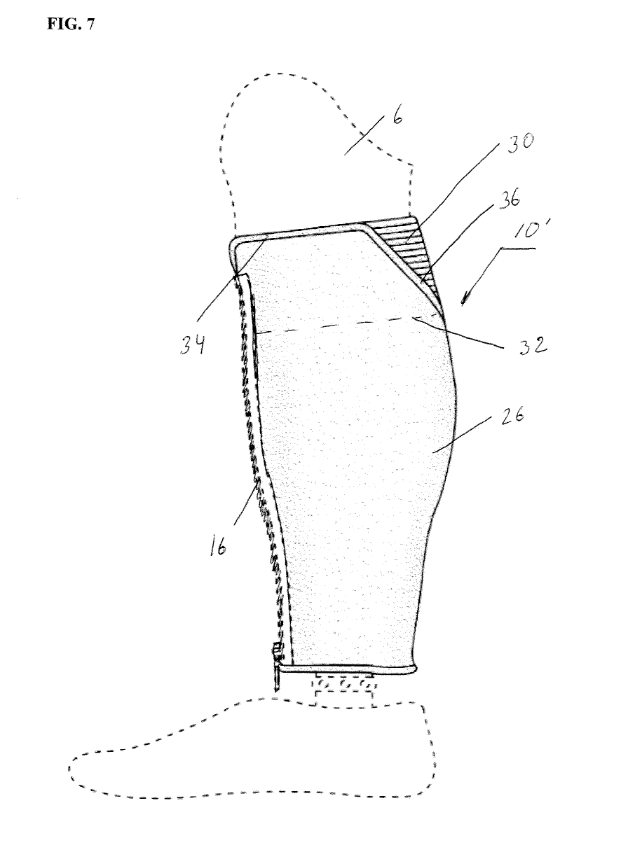

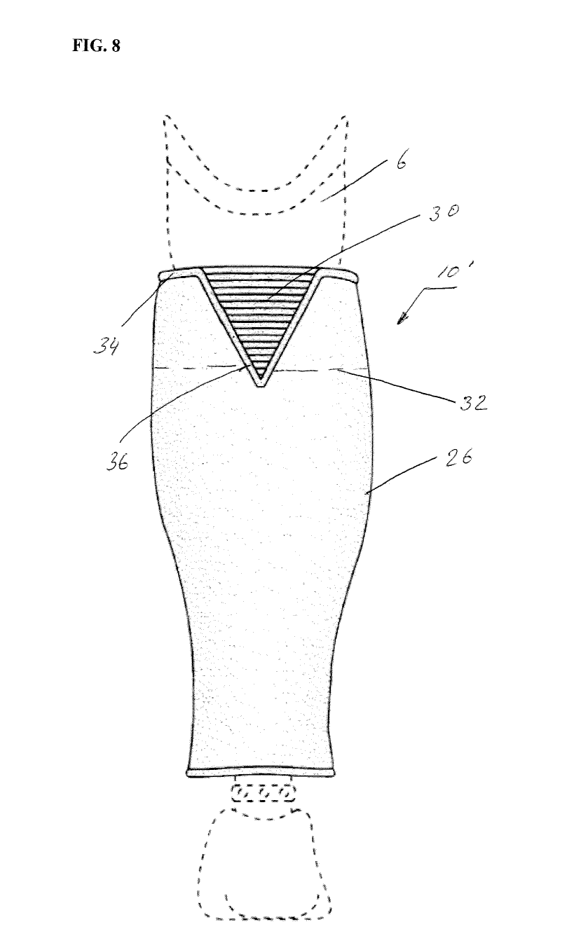

The preferred embodiment of the BK cover 10′ is shown in FIGS. 7 and 8. Similarly, to the above-disclosed cover 10, cover 10′ includes outer layer 26, inner layer 28 (not shown in FIGS. 7 and 8), and zipper 16. Because the foam insert 20 for the BK cover is shorter than for the above-disclosed AK cover, the inner layer is preferably shorter than the outer layer i.e., reaching only to the level 32 located below the top edge 34 of the outer layer. To allow for a smooth transition between the foam portion of the cover 10′ and the socket 6, the outer layer 26 is preferably provided with an expandable elastic section 30. Elastic section 30 is preferably secured within a slit 36 formed in the top portion of the outer layer. The slit 36 is preferably about 3″ long. The elastic section allows the outer layer of the cover 10′ to expand or contract thus accommodating various sizes of the socket and allowing for a smooth transition between the foam portion of the cover and the socket. While the inclusion of the elastic section 30 and shortening of the inner layer 28 are described herein with respect to the BK cover 10′, it should be understood by a person skilled in the art that the same modifications can be incorporated into the cover 10 or any other AK or BK cover disclosed herein.

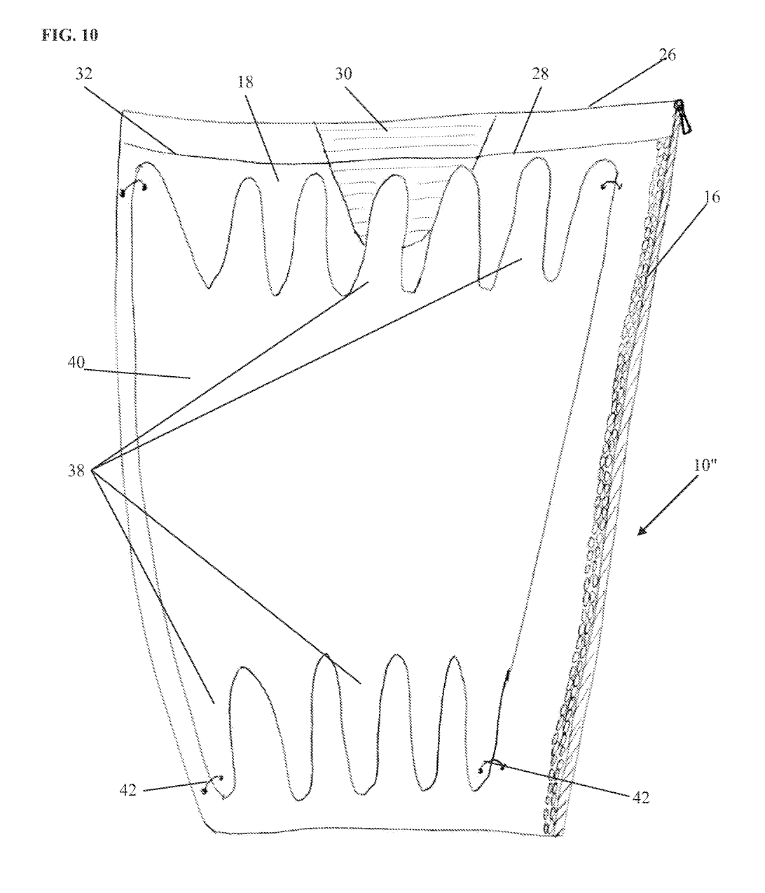

A further preferred embodiment of the cover 10″ is shown in FIG. 10. Similarly to the covers 10 and 10′, cover 10″ includes outer layer 26, inner layer 28 reaching to the level 32 and zipper 16. Further, cover 10″ also includes the expandable elastic section 30. Additionally, cover 10″ includes a bendable layer 40 inserted into the pocket 18 formed by the inner and outer layers. Bendable layer 40 is preferably made of very thin plastic material and includes a plurality of finger-like extensions 38 located at the top and bottom of the bendable layer 40. Layer 40 is preferably secured with stitching 42 to the inner surface of the outer layer 26. Thus, when the foam insert 20 is inserted into the pocket 18, it is located between the inner layer 28 and the bendable layer 40. Other means of securing the bendable layer 40 to the inner surface of the outer layer can be utilized. The layer 40 preferably extends the entire length of the inner layer 28, and, correspondingly, the entire length of the foam insert 20. Provision of the bendable layer 40 allows for a smooth transition between the wider portion of the cover (i.e., at the socket 6) to the narrower portion (i.e., at the pylon 4), thus providing a more realistic appearance and comfort for the user.

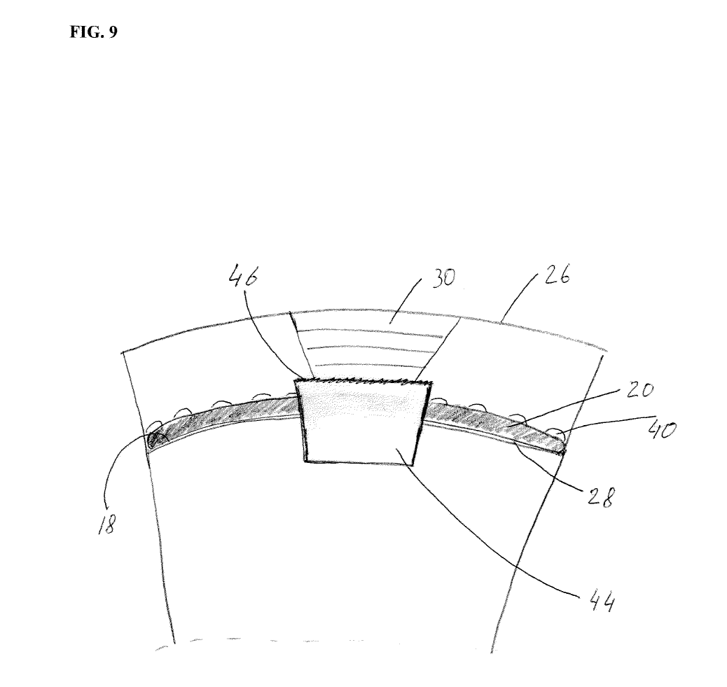

As shown in FIG. 9, to prevent shifting of the foam layer 20 inside the pocket 18, a securing flap 44 is preferably permanently attached to the inner surface of the outer layer 26 with stitching 46. Securing flap 44 extends over the pocket 18 with all of the inserted layers and removably attaches to the inner layer 28, for example, with a hook-and-loop attachment. Thus, in use, the wearer will insert the foam layer 20 into the pocket 18, and then extend the flap 44 over the pocket and attach it to the inner layer 28, securing the foam inside Instead of providing the flap 44, the pocket 18 with its inserts can also be secured by simply attaching the inner layer 28 to the inner surface of the outer layer 26 with a hook-and-loop closure or any other similar attachment.

In the preferred embodiments, the outer layer 26 is made of ⅛ inch thick, latex-free neoprene material. This material is durable, cool to the touch, flexible and can be machine-washed or even washed in a dishwasher. Neoprene does not nick or dent when banged around like the plastic, metal or foam shaped polyurethane products. It does not fade over time in color as well like the painted covers do. Neoprene also stretches. Accordingly, when the leg bends, the joint does not get obstructed as happens with current hard covers. It is also a cheap and durable material. Additionally, many amputees that do not wear covers complain about the pointy and sharp parts of their artificial legs ripping their pants. The neoprene covers stretch over the pointy parts keeping them smooth. The inner layer 28 is preferably made of a net-like elastic lining material. Thus, the entire cover is very lightweight, weighting approximately 12 oz.

The disclosed cover shapes itself to most artificial limbs because of the use of the foam insert. Foam conforms to any artificial leg on the market due to the soft interface. The foam insert by itself is very fragile. Prosthetists sometimes coat the foam with a hard spray but that also nicks when hit and gets holes in it. In the present invention, the foam insert is positioned inside the pocket, which protects it from outside elements. The pocket holds the shape of the foam insert, allowing the insert to function to the best of its ability.

The only covers currently in existence are model specific. They only fit the brand of the artificial legs that makes them. The presently disclosed covers are universal due to the fact that the foam insert has soft attributes that will shape to whatever leg it encloses, once the insert is placed into the pocket and zipped on. Additionally, as shown in FIGS. 4-5, in the presently disclosed covers, the foam insert is pre-shaped to look like a calf and is then inserted into a provided pocket. This feature is not currently available in any existing covers. Other permanent covers are hollowed out and forced over the limb and then painstakingly shaped by hand to match the patient's good leg. The present method has taken out the time it takes to shape the calf because it has off-the-shelf thicknesses and lengths.

In addition to the above-described features, the cover may also include a rubber (shark skin) material at the inside top of the outer layer 26 so as to prevent the cover from spinning around the leg.

All presently disclosed covers are trendy and functional easy-on, easy-off prosthetic limb covers having the following features:
Durable neoprene cool flex outer material which is machine washable and/or can be placed on top shelf of a dishwasher.
Protects the user's investment and replicates a healthy leg.
Prevents dirt and water from affecting the artificial limb.
Provides a lightweight cover.

In the preceding specification, the invention has been described with reference to specific exemplary embodiments thereof. It will however, be evident that various modifications and changes may be made thereto without departing from the broader spirit and scope of the invention. The specification and drawings are accordingly to be regarded in an illustrative manner rather than a restrictive sense.

What is claimed is:

1. A cover for a prosthetic leg comprising:
an outer layer having at least two side edges and an inner surface;
an inner layer permanently attached to the outer layer at said two side edges so as to form a pocket between the inner surface of the outer layer and the inner layer, said inner layer having a top edge;
a foam insert shaped as a partial human leg, said foam insert having an inner cavity and an outer surface, said foam insert being positioned inside said pocket such that said inner cavity of said foam insert is located adjacent to said inner layer, said outer surface of said foam insert faces said inner surface of said outer layer, and said foam insert in its entirety is located below said top edge of said inner layer; and
a closing mechanism attached to the two side edges and operable to selectively and securely attach said side edges to each other.

2. The cover according to claim 1, wherein said outer layer further comprises a top portion, a slit formed in the top portion and an expandable section secured within the slit, and wherein said expandable section allows said top portion to expand and contract.

3. The cover according to claim 2, wherein said outer layer comprises a second top edge, and wherein said top edge of the inner layer is positioned below said second top edge of said outer layer such that said expandable section is located between said top edge of said inner layer and said second top edge of said outer layer.

4. The cover according to claim 1, further comprising a bendable layer positioned within said pocket between said foam insert and said inner surface of said outer layer, said bendable layer having a top portion, a bottom portion and a plurality of firmer-like extensions formed within said top portion and said bottom portion.

5. The cover according to claim 4, wherein said bendable layer is permanently secured to said inner surface of the outer layer.

6. The cover according to claim 5, wherein said bendable layer is formed from a thin plastic material.

7. The cover according to claim 1, wherein said inner layer is removably attachable to the inner surface of the outer layer in an area of the top edge.

8. The cover according to claim 1, wherein said foam insert is universally adaptable to a shape of any artificial limb.

9. The cover according to claim 1, wherein said outer layer is formed from a neoprene material.

10. The cover according to claim 9, wherein said inner layer is formed from a mesh-like material.
